(19) United States
(12) Patent Application Publication
(10) Pub. No.: US 2018/0261329 A1
(43) Pub. Date: Sep. 13, 2018

(54) Personalized Health-Information Based on Genetic Data

(71) Applicant: Segterra, Inc., Cambridge, MA (US)

(72) Inventors: Gil Blander, Lexington, MA (US); Margaret Ploch, La Jolla, CA (US); Bartek Nogal, San Diego, CA (US)

(21) Appl. No.: 15/901,144

(22) Filed: Feb. 21, 2018

Related U.S. Application Data
(60) Provisional application No. 62/461,649, filed on Feb. 21, 2017.

(51) Int. Cl. G16H 50/30; G06F 19/18; A61B 5/00; G16H 50/70; C12Q 1/6827

(52) U.S. Cl. CPC G16H 50/30; G06F 19/18; A61B 5/742; C12Q 2600/118; C12Q 1/6827; C12Q 2600/106; G16H 50/70

(57) ABSTRACT

This disclosure relates to technologies for evaluating health, wellness, and fitness parameters, and producing personalized recommendations, e.g., computer-implemented methods of receiving genetic information of a subject and information on a health-related parameter representing a health condition of the subject, and generating an output (e.g., a personalized recommendation) for the subject.

### CLAIM OF PRIORITY

This application claims priority under 35 USC § 119(e) to U.S. Patent Application Ser. No. 62/461,649, filed on Feb. 21, 2017, the entire contents of which are hereby incorporated by reference.

### TECHNICAL FIELD

This disclosure relates to technologies for evaluating health, wellness, and fitness parameters, and producing personalized recommendations.

### BACKGROUND

Since human genome sequencing became widely available, various genomic analysis approaches have interrogated the human DNA for variants that make human beings unique. Much of the variation occurs at sites called single nucleotide polymorphisms (SNPs) where individuals and/or populations can differ by one or more bases, or alleles. Other types of genetic variations include, e.g., copy number variants (CNVs), insertions/deletions (I/Ds), as well as duplications, translocations and inversions. Genome-wide association studies (GWASs) are non-candidate-driven whole genome studies that have the goal of unearthing gene-trait associations based on discovering significantly altered allele frequencies between groups having and groups not having a particular trait or phenotype.

### SUMMARY

In one aspect, this disclosure features a computer-implemented method that includes receiving genetic information of a subject, and receiving information on a health-related parameter representing a health condition of the subject. The method also includes determining, by one or more processing devices, a target range for the health-related parameter based on the genetic information. The method further includes determining that the health-related parameter of the subject is inside the target range. The method can also include generating, by the one or more processing devices in response to determining that the health-related parameter of the subject is inside the target range, an output indicative of an effect of the genetic information on the health-related parameter of the subject, and presenting the output on an output device. In some implementations, the method can include determining that the health-related parameter of the subject is outside the target range; and generating, in response to determining that the health-related parameter of the subject is outside the target range, a recommendation for affecting the health-related parameter of the subject; and presenting the recommendation on the output device. In some implementations, the target range is determined based on a genetic score calculated using the genetic information. The genetic score can also be compared against a distribution of the genetic score derived from a theoretical population.

In another aspect, this disclosure features a computer-implemented method that includes receiving genetic information of a subject and receiving information on a health-related parameter representing a health condition of the subject. The method can also include determining that the health-related parameter of the subject is outside a predetermined range, and responsive to determining that the health-related parameter of the subject is outside the predetermined range, generating a recommendation for affecting the health-related parameter of the subject, wherein the recommendation is generated based on genetic information of the subject. The method can further include presenting the recommendation on an output device. In some implementations, the method can include generating an initial recommendation responsive to determining that the health-related parameter of the subject is outside the predetermined range, and modifying the initial recommendation based on a genetic score calculated using the genetic information. In some implementations, the genetic score is compared against a distribution of the genetic score derived from a theoretical population.

The disclosure also features a computer-implemented method that includes receiving genetic information of a subject, and retrieving representations of one or more rules for the genetic information. The method can also include applying, by the one or more processing devices, the one or more rules to the genetic information to determine a health-related parameter representing a health condition of the subject. The method further includes generating, in response to the determined health-related parameter, a recommendation, and presenting the recommendation on an output device. In some implementations, the health-related parameter representing the health condition of the subject is determined by a genetic score. The genetic score can also be compared against a distribution of the genetic score derived from a theoretical population.

In another aspect, this disclosure features a system that includes a memory device and an analysis engine. The analysis engine includes one or more processing devices, and is configured to receive genetic information of a subject, receive information on a health-related parameter representing a health condition of the subject, and determine, by one or more processing devices, a target range for the health-related parameter based on the genetic information. The analysis engine is also configured to determine that the health-related parameter of the subject is inside the target range, generate, by the one or more processing devices in response to determining that the health-related parameter of the subject is inside the target range, an output indicative of an effect of the genetic information on the health-related parameter of the subject, and present the output on an output device. In some implementations, the analysis engine is further configured to determine that the health-related parameter of the subject is outside the target range, generate, in response to determining that the health-related parameter of the subject is outside the target range, a recommendation for affecting the health-related parameter of the subject, and present the recommendation on the output device. In some implementations, the target range is determined based on a genetic score calculated using the genetic information. The genetic score can be compared against a distribution of the genetic score derived from a theoretical population.

In one aspect, this disclosure also features a system that includes a memory device and an analysis engine. The analysis engine includes one or more processing devices, and is configured to receive genetic information of a subject, receive information on a health-related parameter representing a health condition of the subject, and determine that the health-related parameter of the subject is outside a predetermined range. The analysis engine is also configured to responsive to determining that the health-related parameter of the subject is outside the predetermined range, generate a recommendation for affecting the health-related parameter of the subject, wherein the recommendation is generated based on genetic information of the subject, and present the recommendation on an output device. In some implementations, the analysis engine is further configured to generate an initial recommendation responsive to determining that the health-related parameter of the subject is outside the predetermined range, and modify the initial recommendation based on a genetic score calculated using the genetic information. In some implementations, the genetic score is compared against a distribution of the genetic score derived from a theoretical population.

In another aspect, this disclosure features a system that includes a memory device and an analysis engine. The analysis engine includes one or more processing devices, and is configured to receive genetic information of a subject, retrieve representations of one or more rules for the genetic information, and apply, by the one or more processing devices, the one or more rules to the genetic information to determine a health-related parameter representing a health condition of the subject. The analysis engine is also configured to generate, in response to the determined health-related parameter, a recommendation, and present the recommendation on an output device. In some implementations, the health-related parameter representing the health condition of the subject is determined by a genetic score. The genetic score can be compared against a distribution of the genetic score derived from a theoretical population.

In another aspect, this disclosure also features one or more machine-readable storage devices storing instructions that are executable by one or more processing devices to perform various operations. The operations include instructions for receiving genetic information of a subject, receiving information on a health-related parameter representing a health condition of the subject, and determining, by one or more processing devices, a target range for the health-related parameter based on the genetic information. The operations can also include determining that the health-related parameter of the subject is inside the target range, generating, by the one or more processing devices in response to determining that the health-related parameter of the subject is inside the target range, an output indicative of an effect of the genetic information on the health-related parameter of the subject, and presenting the output on an output device. In some implementations, the operations can further include instructions for determining that the health-related parameter of the subject is outside the target range, generating, in response to determining that the health-related parameter of the subject is outside the target range, a recommendation for affecting the health-related parameter of the subject, and presenting the recommendation on the output device. In some implementations, the operations also include instructions for calculating a genetic score based on the genetic information. In some implementations, the operations further include instructions for comparing the genetic score against a distribution of the genetic score derived from a theoretical population.

In one aspect, this disclosure features one or more machine-readable storage devices storing instructions that are executable by one or more processing devices to perform various operations. The operations include instructions for receiving genetic information of a subject, receiving information on a health-related parameter representing a health condition of the subject, and determining that the health-related parameter of the subject is outside a predetermined range. The operations also include instructions for responsive to determining that the health-related parameter of the subject is outside the predetermined range, generating a recommendation for affecting the health-related parameter of the subject, wherein the recommendation is generated based on genetic information of the subject, and presenting the recommendation on an output device. In some implementations, the operations also include instructions for generating an initial recommendation responsive to determining that the health-related parameter of the subject is outside the predetermined range, and modifying the initial recommendation based on a genetic score calculated using the genetic information. In some implementations, the operations can further include instructions for comparing the genetic score against a distribution of the genetic score derived from a theoretical population.

In one aspect, this disclosure also features one or more machine-readable storage devices storing instructions that are executable by one or more processing devices to perform various operations. The operations include instructions for receiving genetic information of a subject, and retrieving representations of one or more rules for the genetic information. The operations can also include instructions for applying, by the one or more processing devices, the one or more rules to the genetic information to determine a health-related parameter representing a health condition of the subject, generating, in response to the determined health-related parameter, a recommendation; and presenting the recommendation on an output device. In some implementations, the operations can also include instructions for calculating a genetic score based on the genetic information. In some implementations, the operations can further include instructions for comparing the genetic score against a distribution of the genetic score derived from a theoretical population.

Implementations of the above aspects of the technology can also include one or more of the following features.

The genetic information can include genotypes of one or more SNPs. The genetic information can also include information of copy number variants, insertions/deletions, translocations, and/or inversions.

In some implementations, the health-related parameter represents a level of a blood biomarker.

In some implementations, the target range for the health-related parameter is also determined by demographic information of the subject, a lifestyle parameter of the subject, exercise habit of the subject, or personal goal of the subject.

In some implementations, the recommendation is also determined by demographic information of the subject, a lifestyle parameter of the subject, exercise habit of the subject, or personal goal of the subject.

In some implementations, the health-related parameter is serum 25-hydroxyvitamin D (25(OH)D) level, and the genetic information comprises genotypes of rs2282679.

In some implementations, the health-related parameter is serum triglyceride level, and the genetic information comprises genotypes of rs662799.

In some implementations, the health-related parameter is serum LDL cholesterol level, and the genetic information comprises genotypes of rs964184.

In some implementations, the health-related parameter is serum B12 level, and the genetic information comprises genotypes of rs602662.

In some implementations, the health-related parameter is serum triglyceride level, and the genetic information comprises genotypes of one or more SNPs selected from the group consisting of rs1167998, rs673548, rs780094, rs2240466, rs10096633, rs122272004, rs439401, rs17321515, and rs16996148.

In some implementations, the health-related parameter is fasting blood glucose level, and the genetic information comprises genotypes of one or more SNPs selected from the group consisting of rs7708285, rs11715915, rs17762454, rs2657879, rs340874, rs10747083, rs7651090, rs2302593, rs9368222, rs6943153, rs10814916, rs6072275, rs3829109, rs3783347, rs576674, rs4869272, rs11603334, rs174576, rs11619319, rs11607883, rs7903146, rs4502156, rs11708067, rs11039182, rs10811661, rs1280, rs983309, rs780094, rs10885122, rs2191349, rs11558471, rs6113722, rs16913693, rs2908289, rs560887, and rs10830963.

In some implementations, the health-related parameter is mean platelet volume, and the genetic information comprises genotypes of one or more SNPs selected from the group consisting of rs10914144, rs11071720, rs11602954, rs12485738, rs1668873, rs2138852, rs2393967, rs342293, rs6136489, rs647316, rs7961894, and rs893001.

In some implementations, the subject is a male, the health-related parameter is serum testosterone level, and the genetic information comprises genotypes of rs5934505.

In some implementations, the health-related parameter is predisposition to caffeine consumption, and the genetic information comprises genotypes of rs4410790.

In some implementations, the health-related parameter is aerobic exercise capacity or endurance, and the genetic information comprises genotypes of one or more SNPs selected from the group consisting of rs4646994, rs4343, rs1815739, rs1049305, rs1799722, rs12722, rs12594956, rs11549465, rs5219, rs4253778, rs2016520, rs7732671, rs660339, and rs2010963.

In some implementations, the health-related parameter is lactose intolerance, and the genetic information comprises genotypes of rs4988235.

In some implementations, the technologies described herein may provide one or more of the following advantages.

Personalized recommendations and information for a user (e.g., recommendations and information based on one or more of demographics, biological markers, physiological markers, health and lifestyle-related parameters and goals) can be generated and/or updated based on genetic information for the user. In some implementations, this may improve the quality and applicability of the personalized recommendations, for example, by flagging or deleting recommendations that may not significantly affect a particular user due to his/her genetic profile, or adding recommendations that may be particularly suitable for the user due to his/her genetic profile. By using genetic information in determining an optimized or target range for a health-related parameter (e.g., a level of biomarker or a physiological marker), a more accurate personalized information and/or recommendation may be provided to the user. For example, even if the level of a particular biomarker for a given user is outside a range that may be considered a target range (or an “optimized” range) for a different population, the genetic information for the user may indicate that the level is within a range that is typical for a population with similar or analogous genetic traits. In such cases, the user may be informed that his/her levels are not outside the target range and/or any recommendations associated with the particular biomarker may be updated accordingly.

Unless otherwise defined, all technical and scientific terms used herein have the same meaning as commonly understood by one of ordinary skill in the art. The materials, methods, and examples described in the detailed description are for illustrative purposes and not intended to be limiting. All publications, patent applications, patents, sequences, database entries, and other references mentioned herein are incorporated by reference in their entirety. In case of conflict, the present specification, including definitions, will control.

### BRIEF DESCRIPTION OF THE DRAWINGS

FIG. 1 is a block diagram showing an example of a system for generating recommendations and information for an individual based on genetic data in combination with various health-related parameters.

FIG. 2 is an example of a user interface that summarizes the recommendations for the user based on the user's genetic information.

FIG. 3 is an example of a user interface for presenting the genotype-trait association between a genetic variant and a blood biomarker level.

FIG. 4 is an example of a user interface for presenting the genotype-trait association between a genetic variant and a nutritional sensitivity with related user goals and biomarker levels.

FIG. 5 is diagram showing the input and the output of an example of a process that uses the genotype-trait association between a genetic variant and a blood biomarker level affected by diet.

FIG. 6 is a flow chart showing an example of a process of making a recommendation based on a genetic score.

FIG. 7 is diagram showing the input and the output of an example of a process that uses the genotype trait association between a genetic variant and a blood biomarker level.

FIG. 8 is diagram showing the input and the output of an example of a process that uses the genotype trait association between a genetic variant, microbiome data, and lactose intolerance.

FIG. 9A is a graph showing an example distribution of CRP risk score calculated from a real population dataset.

FIG. 9B is a graph showing an example distribution of a weighted genetic score calculated from a theoretical population.

FIG. 10 is a flowchart representing an example process of generating an output.

FIG. 11 is a flowchart representing an example process of generating a recommendation.

FIG. 12 is a flowchart representing an example process of generating a recommendation.

FIG. 13 is a block diagram of an example of a computing system.

### DETAILED DESCRIPTION

With the advent of renewed focus on personal health and wellness, widespread research on nutrition, exercise, lifestyle, supplements, etc. has produced, and continues to produce, numerous studies, research papers, and articles that present health and wellness-related advice. Many of these publications are publically available to individuals seeking to improve their health and performance. However, the results presented in these publications are based on individuals or groups of individuals with specific sets of health-attributes, and it is challenging to evaluate whether a particular advice or study is applicable to a particular individual. In some cases, evaluating the results or advice from a given study for a particular individual may require not only the details of specific health-attributes used in the study, but also specific information on the health status and/or genetic information of the particular individual.

This disclosure relates to leveraging genetic information of a user, including for example, genome-wide association study (GWAS), as well as candidate gene- based, genotype-phenotype (trait) associations for the purpose of providing personalized recommendations toward optimizing health-related parameters, e.g., blood biomarker levels or physiological marker levels. This disclosure is based, in part, on the fact that certain genetic variants such as single nucleotide polymorphisms (SNPs), copy number variants (CNVs), and nucleotide insertion/deletions (I/Ds), significantly associate with the phenotypes/traits (e.g., serum biomarker levels) in genome-wide association studies and can be easily and relatively inexpensively genotyped in individuals via multiple available platforms (e.g., genome sequencing, DNA microarray chips).

The genetic profile, in combination with the individual's blood test results for the relevant blood biomarker, can be used to make individualized lifestyle, exercise, dietary, and/or nutritional supplement recommendations, and/or to inform the individual that she/he has a predisposition for higher (or lower) than population average levels of a particular biomarker (e.g., triglycerides) based on her/his genotype. In some cases, the recommendation and the predisposition for higher (or lower) than population average levels of a particular biomarker can be determined, in part, by a particular genetic variant such as a SNP being either heterozygous or homozygous for the effect allele that is associated with that particular serum biomarker.

In some cases, where a genetic variant's association with a particular biomarker can be expressed quantitatively, for example, as supported by peer-reviewed literature, adjustments to a user's “optimal zone” for the particular biomarker may be determined based on such a relationship. An optimal zone may be defined as a zone within a clinically normal reference range for the biomarker, wherein the zone represents a desirable or target range for an individual user based on the user's gender, age, activity level and other specific characteristics. Moreover, the rules for generating information and/or recommendations can be modified. For example, as more associations between phenotypes and genetic variants are discovered and/or old associations are replicated and/or quantified, these can be included in reinforcing the process for generating information and/or recommendations. Further, human genetic variants, such as SNP-microbiome interactions, can also be used as input variables in order to refine recommendations.

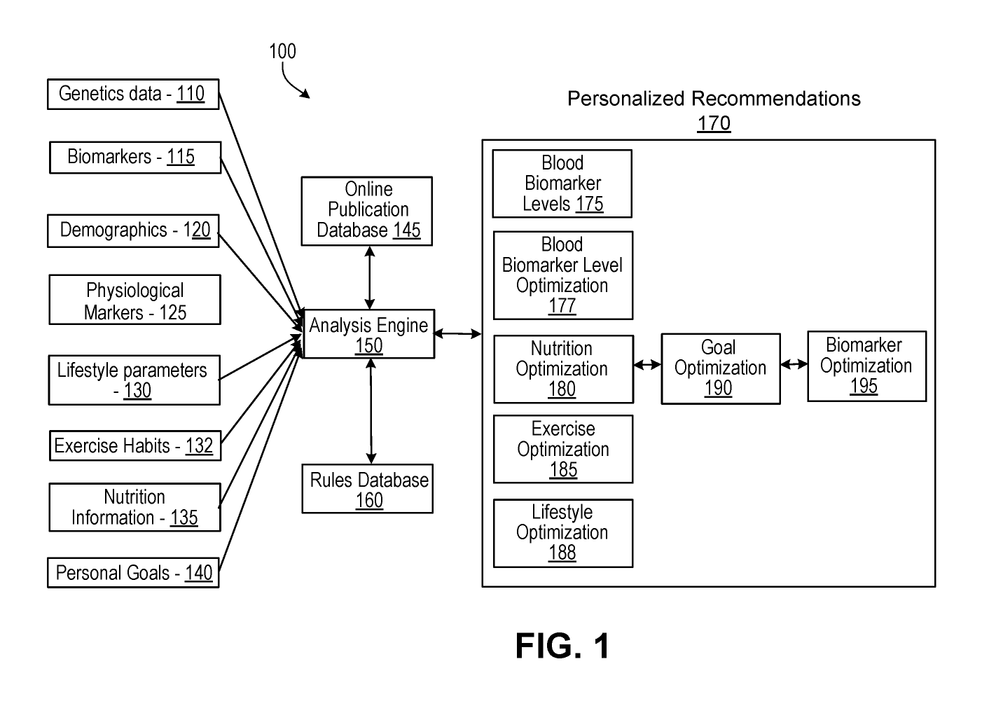

FIG. 1 shows a block diagram of an example of a system 100 that includes an analysis engine 150 for generating recommendations 170 based on a user's genetic data 110 in combination with various other health-related parameters including, for example, biomarkers 115. The analysis engine 150 can also be configured to generate an output (e.g., the personalized recommendations 170) based on various other input parameters including, for example, user demographics 120, physiological markers 125, lifestyle parameters 130, exercise habits 132, nutrition information 135, and/or personal goals 140. In some implementations, the input parameters are based on measurements obtained from a human user. In some implementations, the input parameters can be provided as a representation of a hypothetical individual used in testing and validation.

In some implementations, the analysis engine 150 is in communication with a publication database 145 (e.g., online publication database) and a rules database 160. The analysis engine 150 can be configured to identify one or more rules from the rules database 160, such that the identified rules can be applied to the input parameters, and generate an output (e.g., a personalized recommendation 170). The analysis engine 150 can also be configured to present, for example, on a display device, one or more personalized recommendations 170. The personalized recommendations 170 can include, for example, one or more recommendations related to blood biomarker levels 175, blood biomarker level optimization 177, nutrition optimization 180, exercise optimization 185, lifestyle optimization 188, goal optimization 190, and biomarker optimization 195.

In some implementations, recommendations related to blood biomarker levels 175 can include an output informing a user that the genetic profile of the user places the user in a high, average, or low risk group for an out-of-range (e.g., elevated) blood biomarker level (e.g., hsCRP level). In some cases, the recommendations can also include recommended lifestyle changes, nutrition, and/or exercise in order to reduce the risk. Recommendations related to blood biomarker level optimization 177 can include an output informing the user how well the user may respond to some changes (e.g., take supplements, change lifestyles, do more exercise) in order to optimize the blood biomarker level. For example, if the user has the GT genotype for the SNP rs2282679, the output may inform the user that the long-term response to high-dose vitamin D supplementation may be 9% less efficient. In such cases, the user may be encouraged to take more vitamin D supplementation, increase exposure to sunlight, and take more outdoor activities. Recommendations related to nutrition optimization 180 can include an output informing the user how the user is likely to respond to nutrition. For example, if the user is heterozygous or homozygous for effect allele for rs662799 and the triglyceride level is above the optimized limit, the recommendations may include an output indicating that the triglyceride level is more likely to be elevated when the user consumes omega-6 fatty acid, and suggesting the user consider reducing the amount of omega-6 fatty acids in the diet to improve the triglycerides level. Recommendations related to exercise optimization 185 can include an output informing the user how to optimize exercise routines. For example, if a male user has a high score for TG-GPS and low aerobic activity, the output can inform the user that the user's triglyceride genetic score places the user in the higher-risk group for elevated serum triglyceride level, and that the user may be able to reduce the serum triglyceride level by increasing the level of cardiorespiratory fitness through aerobic exercise. Recommendations related to lifestyle optimization 188 can include an output informing the user how to optimize lifestyle (e.g., bedtime routine). For example, if a user has TT genotype for rs324981, the output can inform the user that the user has a genetic profile that may be associated with how late the user goes to sleep and how long the user stays asleep, and thus, in some appropriate cases, the user should go to bed 30 minutes later and/or sleep 20 min less than those who do not have this genotype. Recommendations related to goal optimization 190 can include an output informing the user how to adjust various factors to achieve the goal. For example, if a user has at least one effect allele (G) for rs5062, the output can inform the user that the user is more likely to gain weight when the user consumes too much saturated fat than those who do not have this genotype, thus, in some appropriate cases, the user should consume less saturated fat. Recommendations related to biomarker optimization 195 can include an output informing the user how various biomarker are optimized when the user tries to achieve his/her goal. For example, if a user has the effect allele for SNP rs4410790 that increases the likelihood that user will consume more caffeine, and the user has a goal to sleep better, the user may receive a recommendation to drink less coffee. The output may further inform the user that by improving the sleep, the user can also improve level of various biomarkers (e.g., glucose level).

The various input parameters can be obtained from various sources. For example, information about the genetic data 110, biomarkers 115, and physiological markers 125 can be obtained from labs or medical records. The genetic data can include genetic information represented by, e.g., SNPs, CNVs, I/Ds, duplications, translocations, inversions, and epigenetics. In some implementations, information about the genetic data 110, various biomarkers 115, demographics 120, physiological markers 125, lifestyle parameters 130, exercise habits 132, nutrition information 135, and/or personal goals 140 can be obtained or provided as inputs from one or more devices such as wearable devices (e.g., smart watches or activity trackers), mobile computing devices (cell phones, tablets) or personalized computer devices (e.g., laptops). Such wearable devices can be configured to measure, compute, or otherwise provide information on various health/fitness related parameters such as heart rate, calorie consumption, calorie expenditure, distance walked, steps taken, electro cardiogram (ECG), or quality of sleep. The devices can also include non-wearable devices such as weight scales or other scales configured to provide information on weight, body-mass index (BMI), or water content of the body.

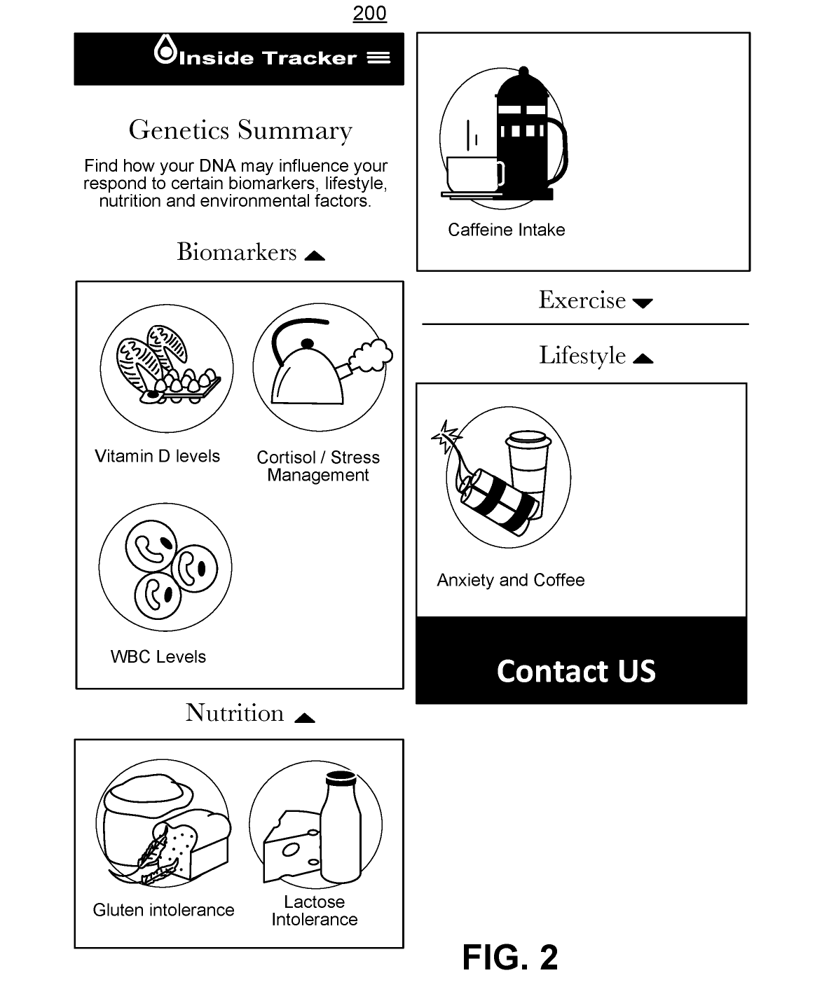

In some implementations, the various input parameters can also be provided to the analysis engine 150 using, for example, personal computer devices or mobile computing devices configured to present an interface for a user to enter the various input parameters. In some implementations, the interface can include the user interface as shown in FIG. 2. In some implementations, the interface can be provided using one or more applications for measuring at least a portion of the various input parameters. In some implementations, the various input parameters can be based on user-input on one or more tests such as blood tests, urine test, sputum test, stool test, or other tests for determining levels of one or more biomarkers 115, genetics data 110 (e.g., SNPs, CNVs, I/Ds, duplications, translocations, inversions, and epigenetics), physiological markers 125, and lifestyle parameters 130. In some implementations, at least a portion of the various input parameters can be obtained from a remote data source. For example, information on these markers and parameters can be obtained from the medical records of the individual based on appropriate permissions from the individual and from a remote storage location (e.g., a cloud storage system) storing such records.

Examples of genetics data 110 can include, e.g., SNPs, CNVs, I/Ds, duplications, translocations, inversions, RNA expression (e.g., whole blood transcript level) and epigenetics. In some implementations, the genetics data can include SNPs in which a single allele is changed to a variant allele, or in which an allele is added or deleted. For example, a variant on one gene may be associated with greater chance for abdominal weight gain for an individual. Other examples of genetic data include SNPs associated with food sensitivity, SNPs that may influence athletic activity and performance, SNPs that may influence particular goals, such as weight loss, better sleep, and/or injury prevention, and SNPs that are associated with overall wellbeing of individuals. The genetics data can be obtained from various sources, e.g., from labs, or medical records.

Examples of health-related parameters include various biomarkers 115, physiological markers 125, lifestyle parameters 130, exercise habits 132, nutrition information 135, and personal goals 140. Examples of biomarkers 115 can include, e.g., glucose, total cholesterol, high density lipoprotein (HDL), low density lipoprotein (LDL), triglycerides, testosterone, free testosterone, estradiol, dehydroepiandrosterone-sulfate (DHEA-S), prolactin, vitamin D, hemoglobin, calcium, parathyroid hormone (PTH), insulin-like growth factor such as IGF-1, tumor necrosis factor (TNF) such as TNF-alpha, pro-inflammatory cytokine such as IL-6, C-reactive protein (CRP), high sensitivity CRP (hsCRP), folic acid, vitamin B12, alanine aminotransferase (ALT), aspartate aminotransferase (AST), gamma-glutamyl transpeptidase, blood urea nitrogen (BUN), ferritin, sodium, zinc, white blood cells, potassium, creatine kinase (CK), sex hormone binding globulin (SHBG), cortisol, albumin, total iron binding capacity (TIBC), unsaturated iron binding capacity (UIBC), progesterone, luteinizing hormone, follicle-stimulating hormone, chromium, thyroid-stimulating hormone (TSH), and magnesium and markers that are part of a Complete Blood Count test, including, for example, hematocrit, mean cell hemoglobin, mean cell hemoglobin concentration, mean cell volume, red blood cell count, red blood cell distribution width, platelets, mean platelet volume, neutrophils (absolute), lymphocytes (absolute), monocytes (absolute), eosinophils (absolute), basophils (absolute), neutrophils (percent), lymphocytes (percent), monocytes (percent), eosinophils (percent), basophils (percent). In some implementations, a genetic marker 123 or telomere length can be used as a biomarker 115.

Examples of demographic parameters 120 can include, e.g., age, gender, and ethnicity. Examples of physiological markers 125 can include, e.g., heart rate variability, pulse pressure, heart rate, BMI, blood pressure, weight, body fat percentage, and height. Examples of lifestyle parameters 130 can include activity level (e.g., amount of exercise done per day), smoker status (e.g., whether or not a smoker, time since quitting, number of cigarettes or other units smoked per day etc.), lactose intolerance, and user behavior. Examples of user behavior can include, e.g., one or more of: number of caffeinated beverages consumed daily, frequency of consuming certain types of food such as dairy and red meat, type and amount of dietary supplements taken, and time spent in the sun daily. Examples of exercise habits 132 can include, e.g., activity level (e.g., amount of exercise done per day), the exercise routine for the subject (e.g., cardiovascular exercise, strength training), time of the exercise, and exercise training program. Examples of nutrition information 135 can include, e.g., the amount of protein, carbohydrate, calories, fat (e.g., saturated or unsaturated), vitamins (e.g., vitamin A, C, D, E, K, B6, B12), calcium, ion, magnesium, folic acid, or some other supplementary nutrition in the food taken by the subject. Examples of personal goals 140 can include, e.g., user-defined objectives such as to sleep better, improve immune function, lose fat, gain muscle, maintain weight, build strength and power, build endurance, prevent injury/speed recovery, reduce stress, fight aging, sleep better, strengthen immune system, improve cognition, boost energy, improve digestion, improve sex life, improve bone health and/or increase endurance.

In some implementations, the analysis engine 150 can be configured to process genetic information of a subject and information on a health-related parameter representing a health condition of the subject and provide an output indicative of an effect of the genetic variants on the health-related parameter of the subject. In some implementations, the analysis engine 150 can be configured to generate one or more recommendations for affecting the health-related parameter of the subject.

In some implementations, the rules are applied to a genetic score derived from the genetic information. In some implementations, the genetic score can be a weighted genetic score or an unweighted genetic score. In some implementations, a weighted genetic score or weighted genetic potential score (e.g., triglyceride genetic potential score (TG-GPS), fasting glucose genetic potential score (FG-GPS)) can be calculated using a formula such as:

Genetic Score=(Scaling Factor)×[SNP₁×Effect₁+SNP₂×Effect₂+ . . . SNPₙ*Effectₙ]
where SNPₙ refers to the genotype for a particular rsID and is given a value of 0, 1, or 2, depending on whether a subject is homozygous for the non-effect allele (0), heterozygous (1), or homozygous for the effect allele (2). The effect sizes can be fixed, and in some cases, equal to linear regression coefficients or similar weights in published studies (in some cases, a meta-analysis study). The scaling factor is a number which scales (or multiplies) the sum of the product of SNP value and effect size to a determined range. In some implementations, the scaling factor is 1. In some implementations, the scaling factor is selected to map a value into a predetermined range, e.g., [0,100].

In some implementations, an unweighted genetic score or unweighted genetic potential score can be calculated based on the following formula:

Genetic Score=[SNP₁+SNP₂ . . . SNPₙ]

wherein SNPn refers to the genotype for a particular rsID and is given a value of 0, 1, or 2, depending on whether a user is homozygous for the non-effect allele (0), heterozygous (1), or homozygous for the effect allele (2). In some cases, the effect allele of each SNP has the same or similar effect to the health-related parameter of interest (e.g., increase the health-related parameter).

In some implementations, one or more datasets that represent a theoretical population can be generated. A theoretical population is a simulated population in which each subject in the population is generated by appropriate rules, e.g., Hardy-Weinberg equilibrium. For example, if the frequency of the effect allele (designated as “A”) is p, then the frequency of the non-effect allele (designated as “B”) is 1-p. The frequency of effect allele can be determined from NCBI SNP database. According to Hardy-Weinberg equilibrium, the expected frequency for genotype AA is p², the expected frequency for genotype AB is p(1-p), and the expected frequency for genotype BB is (1-p)². Thus, a theoretical population can be generated by randomly assigning p² proportion of the subjects in the population with the AA genotype, assigning p(1-p) proportion of the subjects in the population with the AB genotype, and assigning (1-p)² proportion of the subjects in the population with the BB genotype. This procedure can be repeated for each SNP of interest. A genetic score (e.g., weighted genetic score or unweighted genetic score) can be calculated for each subject in the theoretical population. In some implementations, the distribution of the genetic score can be compared against the distribution of the health-related parameter (e.g., serum triglyceride level) derived from a real population. If the distribution of the genetic score is substantially similar to the distribution of the health-related parameter derived from a real population, then the genetic score may be considered as validated.

In some implementations, the genetic score for a subject is placed in the genetic score distribution derived from the theoretical population in order to determine the percentile of the subject's genetic score within the theoretical population. The analysis engine 150 can then generate an output (e.g., a recommendation) based on the percentile of the subject's genetic score within the theoretical population. For example, if the triglyceride genetic potential score (TG-GPS) for a subject is placed in 90% percentile in the distribution (higher than 90% of the subjects in the theoretical population) and has high serum triglyceride level, the analysis engine 150 will generate an output (e.g., a recommendation), recommending him to increase his level of cardiorespiratory fitness in order to modify his serum triglyceride level. The recommendations can also be adjusted by the exact percentile rank for the subject's genetic score. For example, the recommended level of cardiorespiratory exercise can be adjusted by the percentile rank for the subject's genetic score. A higher percentile rank usually indicates a higher level of cardiorespiratory exercise is required. In some implementations, the analysis engine 150 is configured to predict a quantitative serum biomarker shift within the normal clinical range based on a particular genetic variant or combination of genetic variants. In some implementations, the analysis engine 150 is configured to predict serum biomarker in response to nutritional supplements based on a particular genetic variant or combination of genetic variants. In some implementations, the analysis engine 150 is configured to predict an individual's serum biomarker in response to a particular dietary intervention based on a particular genetic variant or combination of genetic variants. In some implementations, the analysis engine 150 is configured to predict an individual's serum biomarker in response to a particular environmental stimulus based on a genetic variant or combination of genetic variants. In some implementations, the analysis engine 150 is configured to adjust an individual's personalized serum biomarker “optimal zone” within the normal clinical reference range based on a particular genetic variant or combination of genetic variants. In some implementations, the analysis engine 150 is configured to substantiate predisposition to a nutritional deficiency (or lack thereof) through the combination of serum biomarker results and a genetic variant or combination of genetic variants associated with the biomarker.

In some implementations, the analysis engine 150 is configured to substantiate predisposition to altered biomarker levels through the combination of serum biomarker results and a genetic variant or combination of genetic variants associated with said biomarker. In some implementations, the analysis engine 150 is configured to inform the user about his/her genetic predisposition to having higher (or lower) serum biomarker levels relative to the population average through a weighted personal genetic score based on multiple genome-wide significant DNA variants, and, combined with said user's relevant serum biomarker results, generate recommendations (when available based on peer-reviewed literature) toward modifying genetic risk of altered levels of the biomarker.

In some implementations, the analysis engine 150 can also be configured to access data for a plurality of input parameters that includes one or more goals of an individual, and identify a set of one or more out-of-range parameters or unmet goals. The analysis engine 150 can be further configured to identify one or more rules from the rules database 160, such that the identified rules are related to improving the corresponding levels of the one or more out-of-range parameters. The analysis engine 150 can also be configured to present, for example, on a display device, one or more personalized recommendations 170. The personalized recommendations can include, for example, one or more recommendations related to exercises, lifestyle, diet, supplement, and educational materials. In some implementations, the recommendations may be provided on a unified user interface presented on a display device.

In some implementations, the analysis engine 150 can be configured to augment the rules database 160 by facilitating creation of one or more new rules based on new results/studies that may become available. Evidence of scientific support for the new results/studies can include, for example, publications such as peer-reviewed research articles, technical papers or datasheets, publications from government agencies such as the National Institutes of Health, and publications from regulatory bodies such as the US Olympic Committee. In some implementations, the analysis engine 150 can be configured to retrieve one or more attributes of a particular publication based, for example, on an identifier associated with the particular publication. For example, the analysis engine can be configured to receive, e.g., via a user interface, an identifier associated with a publication, and retrieve the one or more attributes from a corresponding publication database 145. The publication database 145 can be identified, for example, based on the identifier associated with the publication. In some implementations, the publication database 145 is stored on a remote storage device, and connected to the analysis engine 150 over a network (e.g., the Internet). In some implementations, the publication database can be stored on a local storage device (e.g., a storage device that also stores the rules database 160).

In some implementations, the analysis engine 150 can be configured to implement a review process that can be used to ensure, for example, that the rules are unambiguous, accurate, and supported by research evidence. Using such a process, various personnel including scientists are able to review a rule (possibly via multiple iterations) before the rule is finalized. For example, the analysis engine can present an interface that allows a scientist to define a rule and save a draft version of the rule on a storage device. The analysis engine 150 can also be configured to present a reviewable version of the rule, for example, to a person with approval capacity. This can be done, for example, upon verifying by the analysis engine 150, that the person indeed has approval capacity. In some implementations, the analysis engine 150 may have access to a database of authorized users and their respective levels of permissions. The analysis engine 150 may use information from such a database to determine if a person has permissions to review, approve and/or lock a given rule. In some implementations, the permissions can be specific to a rule or type of rule. In some implementations, one or more administrative users can have permissions to add new users, set and change permission levels, and delete users.

The approver may evaluate various components of the rule (including, for example, the publication source associated with the rule) via the interface presented by the analysis engine. Upon receiving an indication of approval, the analysis engine can be configured to change a status of the rule accordingly. Alternatively, upon receiving notification that one or more edits are to be made, the analysis engine 150 can be configured to change the status of the rule to indicate a draft status, and present the rule in an editable interface for a personnel (e.g., the original author) to make any edits. Approved rules can be presented by the analysis engine for a final review and “locked” to prevent any inadvertent changes. In some implementations, the analysis engine 150 can only use locked rules to generate recommendations for an end-user. If a locked rule needs to be changed for any reason, the analysis engine can present an administrator interface for unlocking the rule for editing, and updating the status of the rule to a draft status.

In some implementations, a rules database 160 can be used to generate personalized advice on nutrition, exercise, lifestyle, supplement and other health-related issues. Rules from the database can be personalized for individuals (or group of individuals), based on specific inputs on one or more parameters associated with the individual (or group of individuals). Examples of such parameters include biomarkers, physiological markers, demographics, health and lifestyle parameters, and personal goals of the individuals. In one illustrative example one parameter (e.g., a biomarker like vitamin D) may affect one or more of other parameters. For example, low vitamin D may affect testosterone levels in men and bone health in both sexes. In another example, vitamin D production in the skin may vary with ethnicity such that individuals of one particular ethnicity (e.g., African-Americans) may need to consume more vitamin D in supplement form to reach a predetermined optimal level. This disclosure describes technology for managing and augmenting a rules database based on new studies and results as they become available. The technology facilitates creating and editing a library of sources that may be used to support present or future rules. The technology also enables users to create source entries that include a variety of data that may be used to sort and rank the rules, for example, based on the corresponding publication sources.

Various relationships between the rules and the corresponding sources can be used within the rules database, including, for example, one-to-one, one-to-many, many-to-one, and many-to-many. For example, a single source may be used to support one or more rules, and/or one or more sources may be used support a single rule. For example, a single publication may serve as the basis for a rule that suggests taking 3-5 mg melatonin to facilitate sleep in a new time zone, as well as for another rule that suggests taking meals 1-2 hours earlier than usual when traveling eastwards or 1-2 hours later than usual when traveling westwards. On the other hand, a rule that suggests using earplugs or other means of blocking out noise in order to improve sleep quality may be supported by two separate sources (e.g., a research article as well as the USOC Jet Lag Countermeasures and Travel Strategies Brochure).

In some implementations, management of the rules database is facilitated by providing an interface that allows for controlling an expert system without having to reprogram the system. This allows for health experts such as biologists, exercise physiologists, nutritionists, and other scientists to create new rules, simulate specific conditions (e.g., as represented by a set of parameters of an individual) to test validity and/or applicability of the rules, and refine the rules, for example, to tailor the rules for the specific conditions. In some implementations, the technology described herein includes a version control system that checks for inconsistencies and redundancies, and replaces outdated rules with more recent versions. Additional description on adding, deleting, editing, or otherwise managing the rules in a rules database is described in International Application No. PCT/US2016/061290, filed on Nov. 10, 2016, the entire content of which is incorporated herein by reference.

FIG. 2 shows an example of a user interface 200 summarizing the results of analyzing the genetic information. These results can include various genotype-trait associations for all the genetic variants (e.g., SNPs) analyzed for the user. These genotype-trait associations can be used to determine various health-related parameters, including, e.g., biomarkers, nutrition, exercise, and lifestyle.

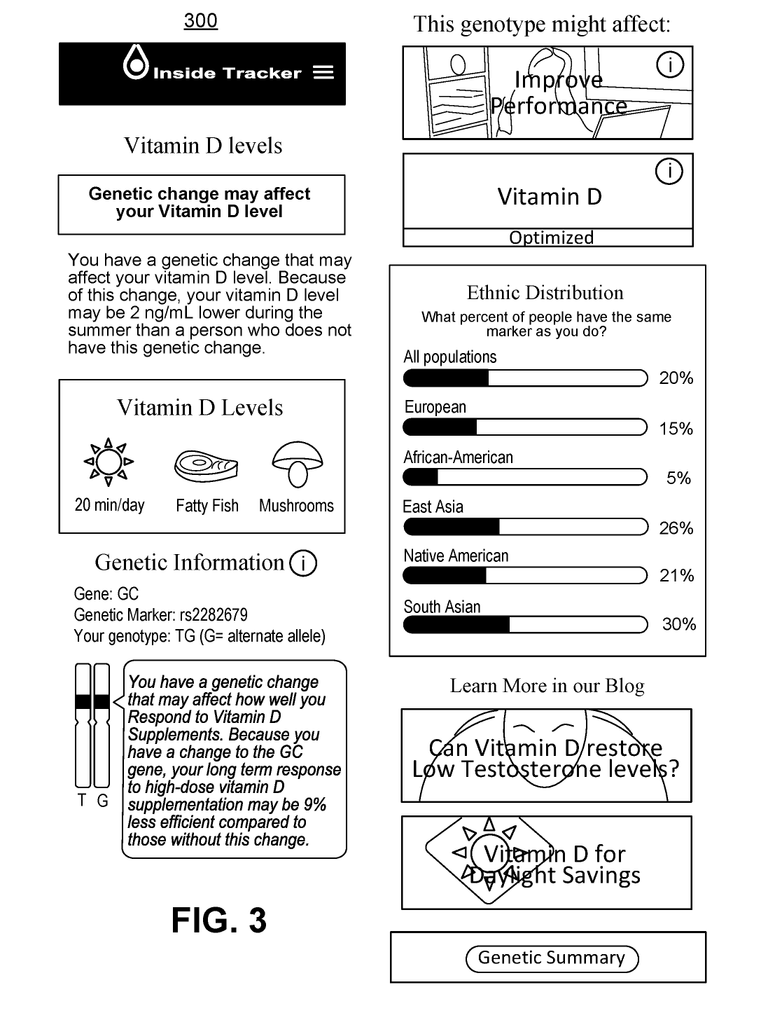

FIG. 3 shows an example of a user interface 300 summarizing the results of analyzing the genotype of SNP rs2282679 (gene: GC; T>G) for a subject. The rule parameters involve the genotype-trait association between rs2282679 and serum 25-hydroxyvitamin D (25(OH)D) levels. The association between rs2282679 and serum 25-hydroxyvitamin D (25(OH)D) levels has been confirmed in multiple independent large-scale studies. For example, the relationships between genetic variants and serum 25-hydroxyvitamin D (25(OH)D) levels and/or other health-related parameters are described in the following references: Moy, Kristin A., et al. “Genome-wide association study of circulating vitamin D-binding protein,” The American journal of clinical nutrition 99.6 (2014): 1424-1431; Perna, Laura, et al. “Genetic variations in the vitamin D binding protein and season-specific levels of vitamin D among older adults,” Epidemiology 24.1 (2013): 104-109; Ahn, Jiyoung, et al. “Vitamin D-related genes, serum vitamin D concentrations, and prostate cancer risk,” Carcinogenesis (2009): bgp055; Wang, Thomas J., et al. “Common genetic determinants of vitamin D insufficiency: a genome-wide association study,” The Lancet 376.9736 (2010): 180-188; Trummer, Olivia, et al. “Allelic determinants of vitamin d insufficiency, bone mineral density, and bone fractures,” The Journal of Clinical Endocrinology & Metabolism 97.7 (2012): E1234-E1240; Cheung, Ching-Lung, et al. “Genetic variant in vitamin D binding protein is associated with serum 25-hydroxyvitamin D and vitamin D insufficiency in southern Chinese,” Journal of human genetics 58.11 (2013): 749-751; and Slater, Nicole A., et al. “Genetic variation in CYP2R1 and GC genes associated with vitamin D deficiency status,” Journal of pharmacy practice (2015): 0897190015585876.Each of the above references is incorporated herein by reference in its entirety.

The relationships between rs2282679 and serum 25-hydroxyvitamin D (25(OH)D) levels may be used to determine a target range of 25(OH)D for a particular individual. For example, if an individual is homozygous for the effect “G” allele (i.e. genotype: GG) for rs2282679, the analysis engine 150 can be configured to determine (e.g., by accessing a relevant rule in the rules database 160) that the serum 25(OH)D level in the individual is 3.6 ng/mL lower than that of individuals having the TT genotype during the summer months. Similarly, if an individual is heterozygous (having genotype GT), the analysis engine 150 can be configured to determine that the serum 25(OH)D level in the individual is 2.0 ng/mL lower that of individuals having the TT genotype during the summer months. Furthermore, since rs2282679 has not been associated with bone mineral density (BMD) and the magnitude of this variation is ˜10% within the normal reference range, the “optimal zone” or “target range” for users with the GT or GG genotype for serum 25(OH)D would be adjusted by 2-3.6 ng/mL to reflect her/his likely achievable genetic limit. The optimal zones of those individuals who are of the TT genotype would not be adjusted based on their genetic information. In some implementations, if a user has the GG genotype for the SNP rs2282679 and has a 25(OH)D blood test result that is below his/her target range, the analysis engine 150 will determine that the user is less responsive (e.g., 14% less responsive) to vitamin D supplementation. In contrast, if the use is heterozygous for rs2282679, the analysis engine 150 may determine that the corresponding user is less responsive (e.g., 9% less responsive) to vitamin D supplementation. In some embodiments, the analysis engine 150 may accordingly generate a personalized recommendation 170 (e.g., taking more vitamin D supplementation, increasing exposure to sunlight, taking more outdoor activities), and display the recommendation on an output device.

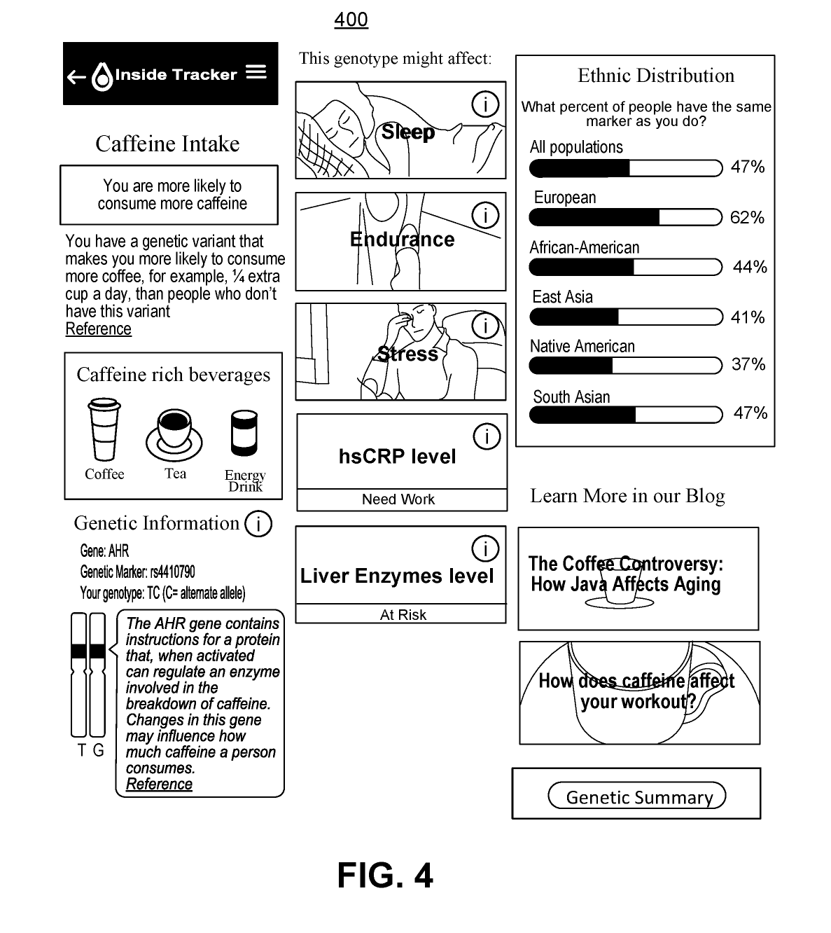

FIG. 4 shows an example of a user interface 400 summarizing the results of analyzing the genotypes of SNP rs4410790. The SNP rs4410790 (gene: AHR; T>C) is associated with predisposition to caffeine consumption. The genotypes of this SNP can affect the user's goals, such as improving sleep and/or biomarker levels (e.g., glucose). The relationship of genetic variants and predisposition to caffeine consumptions is described, e.g., in Cornelis, Marilyn C., et al. “Genome-wide meta-analysis identifies regions on 7p21 (AHR) and 15q24 (CYP1A2) as determinants of habitual caffeine consumption,” PLoS Genet 7.4 (2011): e1002033, which is incorporated herein by reference in its entirety.

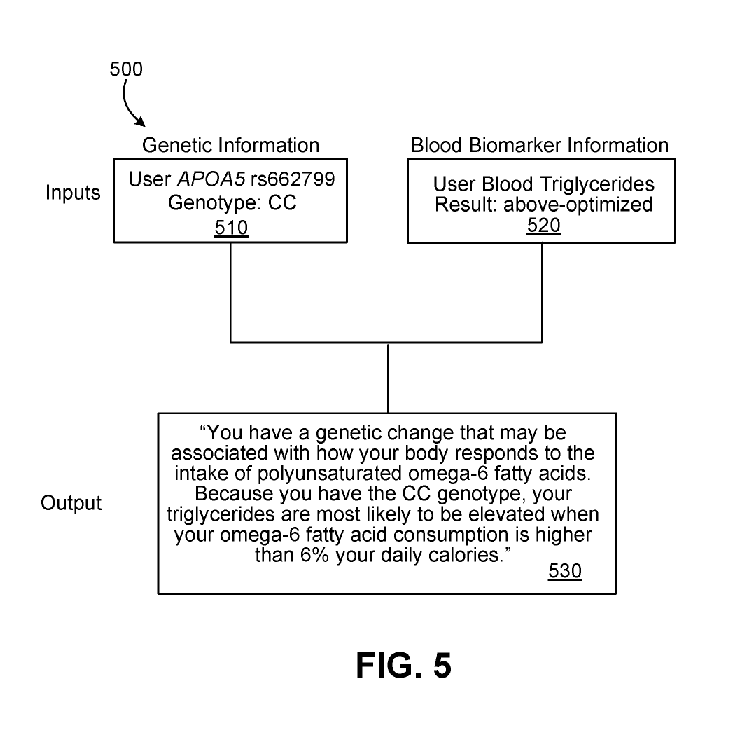

FIG. 5 is a block diagram showing the input and the output of an example of a process based on the genotype-trait association between a genetic variant and blood biomarker levels. In this process, the input is genetic information 510 (e.g., the genotype CC of SNP rs662799 (gene: APOA5; T>C)) and blood biomarker information 520 (e.g., user blood triglyceride result: above-optimized). The analysis engine 150 analyzes the genetic information 510 and the blood biomarker information 520, and provides an output 530 based on the association between rs662799 and blood triglyceride level. In some implementations, if the user has a measurement for triglycerides that is outside his/her optimized zone and is either heterozygous or homozygous for the effect allele (“C”), the user may be informed that she/he is more likely to have high triglycerides due to omega-6 fatty acid consumption of >6% of daily caloric intake. The association between the genetic variants and serum triglyceride level in response to dietary intake of n-6 fatty acids is described, e.g., in Lai, Chao-Qiang, et al. “Dietary Intake of n-6 fatty acids modulates effect of apolipoprotein A5 gene on plasma fasting triglycerides, remnant lipoprotein concentrations, and lipoprotein particle size,” Circulation 113.17 (2006): 2062-2070; and Jang, Yangsoo, et al. “The- 1131T→C polymorphism in the apolipoprotein A5 gene is associated with postprandial hypertriacylglycerolemia; elevated small, dense LDL concentrations; and oxidative stress in nonobese Korean men,” The American journal of clinical nutrition 80.4 (2004): 832-840; each of which is incorporated herein by reference in its entirety.

In some implementations, the input can include the genotypes of rs964184 (gene: ZPR1; C>G) and LDL cholesterol level. In one example, if the user has an elevated LDL cholesterol and is either heterozygous or homozygous for the “G” allele in rs964184, the analysis engine 150 can be configured to generate an output informing the user that the user is more likely to experience reductions in LDL cholesterol level on a low fat as opposed to a higher fat diet. The relationship between genetic variants and the lipid metabolism is described, e.g., in Zhang, Xiaomin, et al. “APOA5 genotype modulates 2-y changes in lipid profile in response to weight-loss diet intervention: the Pounds Lost Trial,” The American journal of clinical nutrition 96.4 (2012): 917-922, which is incorporated herein by reference in its entirety.

In some implementations, the analysis engine 150 is configured to substantiate a user's predisposition to a nutritional deficiency (or lack thereof) through a combination of serum biomarker results and genetic information (e.g., genotypes of SNPs). In some implementations, the genetic information comprises the genotypes of SNP rs602662 (gene: FUT2; G>A). In one example, if a user has above-optimized levels of vitamin B12 in the normal clinical reference range and is either heterozygous (GA) or homozygous (AA) for the “A” allele, the analysis engine 150 can be configured to generate an output informing the user that the user is likely to have serum B12 that is 44 pg/mL higher than those who do not have the “A” allele, and this user is also likely to be B12 deficient on a vegetarian diet. The relationship between genetic variants and plasma vitamin B12 levels is described, e.g., in Hazra, Aditi, et al. “Common variants of FUT2 are associated with plasma vitamin B12 levels,” Nature genetics 40.10 (2008): 1160-1162, which is incorporated herein by reference in its entirety.

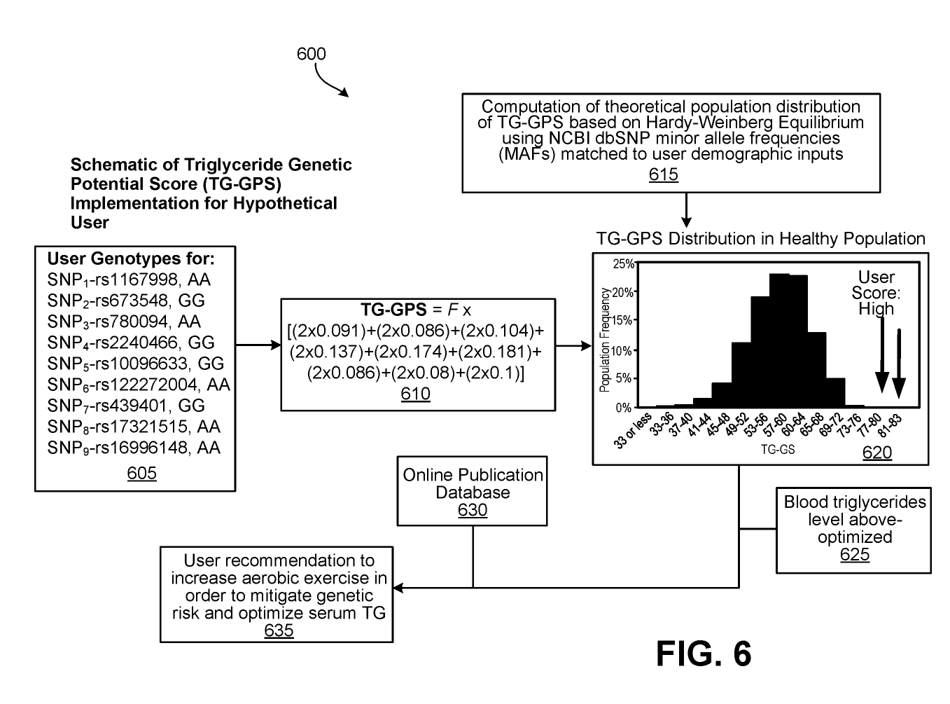

FIG. 6 shows an example of a process 600 for generating a recommendation based on triglyceride genetic potential score (TG-GPS). The recommendation 635 is based on genetic information 605 (e.g., genotypes of multiple SNPs). In this example, the analysis engine 150 is configured to calculate the multi-cohort-based and weighted genetic score TG-GPS based on the rule 610. In some implementations, SNPs that are genome-wide significant for a particular trait (that is, the association p-value is <10⁻⁸) are used to calculate the predisposition of an individual to have “low”, “average”, or “high” levels of a biomarker 620. For example, a serum triglycerides genetic score (TG-GPS) can be determined by the genotypes of rs1167998, rs673548, rs780094, rs2240466, rs10096633, rs122272004, rs439401, rs17321515, and rs16996148, with respective effect sizes of 0.091, 0.086, 0.103, 0.137, 0.174, 0.181, 0.086, 0.08, and 0.1 derived from a meta-analysis that found these SNPs to be genome-wide significant for triglycerides. The relationship between genetic variants and blood biomarkers are described, e.g., in Aulchenko, Yurii S., et al. “Loci influencing lipid levels and coronary heart disease risk in 16 European population cohorts,” Nature genetics 41.1 (2009): 47-55; Kathiresan, Sekar, et al. “Six new loci associated with blood low-density lipoprotein cholesterol, high-density lipoprotein cholesterol or triglycerides in humans,” Nature genetics 40.2 (2008): 189-197; and Tanisawa, Kumpei, et al. “Polygenic risk for hypertriglyceridemia is attenuated in Japanese men with high fitness levels,” Physiological genomics 46.6 (2014): 207-215; each of which is incorporated herein by reference in its entirety.

In some implementations, TG-GPS can be calculated using the formula:

TG-GPS=(Scaling Factor)×[SNP₁×Effect₁+SNP₂×Effect₂+ . . . SNPₙ*Effectₙ],

wherein SNPₙ refers to the genotype for a particular rsID and is given a value of 0, 1, or 2, depending on whether a user is homozygous for the non-effect allele (0), heterozygous (1), or homozygous for the effect allele (2). The effect sizes are fixed and equal to the linear regression coefficients or similar weights in published studies. In some implementations, at least 1, at least 2, at least 3, at least 4, at least 5, at least 6, at least 7, at least 8, or all 9 SNPs selected from the group consisting of rs1167998, rs673548, rs780094, rs2240466, rs10096633, rs122272004, rs439401, rs17321515, and rs16996148 are selected to calculate TG-GPS. In some implementations, in order to place an individual within population distribution of these particular variants, a theoretical population can be generated using Hardy-Weinberg equilibrium 615.

With the individual's calculated TG-GPS calculated and serum TG results 625, a recommendation can be made toward correcting high triglycerides 635. For example, if the user is a male and has high serum triglycerides as well as a high or average TG-GPS, the analysis engine will generate an output (e.g., a recommendation), recommending him to increase his level of cardiorespiratory fitness in order to modify his serum TG's, as published work obtained from online publication database 630 has suggested that a high or average genetic risk of elevated TG's can be modified via increased fitness levels. Of note, recommendations are personalized based on a match between the individual's reported demographics, BMI, age, activity levels, and goals, and the population studied for that particular genotype-serum biomarker recommendation.

In some implementations, the recommendation is based on multiple SNPs incorporated into multi-cohort-based and weighted genetic score for fasting glucose level. In some implementations, the fasting glucose genetic score (FG-GPS) is calculated based on the genotypes of rs7708285, rs11715915, rs17762454, rs2657879, rs340874, rs10747083, rs7651090, rs2302593, rs9368222, rs6943153, rs10814916, rs6072275, rs3829109, rs3783347, rs576674, rs4869272, rs11603334, rs174576, rs11619319, rs11607883, rs7903146, rs4502156, rs11708067, rs11039182, rs10811661, rs1280, rs983309, rs780094, rs10885122, rs2191349, rs11558471 rs6113722, rs16913693, rs2908289, rs560887, and rs10830963, with respective effect sizes of 0.011, 0.012, 0.012, 0.012, 0.013, 0.013, 0.013, 0.014, 0.014, 0.015, 0.016, 0.016, 0.017, 0.017, 0.017, 0.018, 0.019, 0.02, 0.02, 0.021, 0.022 0.022, 0.023, 0.023, 0.024, 0.026, 0.026, 0.027, 0.027, 0.029, 0.029, 0.035, 0.043, 0.057, 0.071, and 0.078, respectively, as derived from a meta-analysis that found these SNPs to be genome-wide significant for fasting glucose level. The relationship between genetic variants and glycemic traits is described, e.g., in Scott, Robert A., et al. “Large-scale association analyses identify new loci influencing glycemic traits and provide insight into the underlying biological pathways,” Nature genetics 44.9 (2012): 991-1005, which is incorporated herein by reference in its entirety. In some implementations, the SNPs for calculating TG-GPS can include, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, or all 36 SNPs selected from the group consisting of rs7708285, rs11715915, rs17762454, rs2657879, rs340874, rs10747083, rs7651090, rs2302593, rs9368222, rs6943153, rs10814916, rs6072275, rs3829109, rs3783347, rs576674, rs4869272, rs11603334, rs174576, rs11619319, rs11607883, rs7903146, rs4502156, rs11708067, rs11039182, rs10811661, rs1280, rs983309, rs780094, rs10885122, rs2191349, rs11558471, rs6113722, rs16913693, rs2908289, rs560887, and rs10830963.

In some implementations, FG-GPS is calculated using the formula:

FG-GPS=(Scaling Factor)×[SNP₁×Effect₁+SNP₂×Effect₂+ . . . SNPₙ*Effectₙ],

wherein SNPn refers to the genotype for a particular rsID and is given a value of 0, 1, or 2, depending on whether a user is homozygous for the non-effect allele (0), heterozygous (1), or homozygous for the effect allele (2). The effect sizes are fixed and equal to the published study linear regression beta coefficients. In order to place an individual within population distribution of these particular variants, a theoretical population can be generated using Hardy-Weinberg equilibrium. Based on the FG-GPS the user's current and historical fasting glucose blood test results, and potentially one or more lifestyle parameters such as diet, the analysis engine 150 can be configured to generate a recommendation for the user such that the recommendation may be usable for affecting fasting glucose levels to move the levels into a target range. Because gut microbiome may influence fasting glucose levels, in some implementations, the analysis engine 150 can be configured to generate the recommendation based on information on a user's gut microbiome.

In some implementations, the recommendation is based on multiple SNPs incorporated into multi-cohort-based but unweighted potential score. The unweighted genetic potential score for MPV-GPS can be calculated based on the following formula:

MPV-GPS=[SNP₁+SNP₂++SNPₙ],

wherein SNPₙ refers to the genotype for a particular rsID and is given a value of 0, 1, or 2, depending on whether a user is homozygous for the non-effect allele (0), heterozygous (1), or homozygous for the effect allele (2). These SNPs can include at least 1, at least 2, at least 3, at least 4, at least 5, at least 6, at least 7, at least 8, at least 9, at least 10, at least 11 or all 12 SNPs selected from the group consisting of rs10914144, rs11071720, rs11602954, rs12485738, rs1668873, rs2138852, rs2393967, rs342293, rs6136489, rs647316, rs7961894, and rs893001. Here, each additional effect allele (i.e. one (1) genetic score unit) may add a predetermined amount (e.g., 0.12 fL) to the MPV blood test reading within the normal clinical reference range and, provided an rsID has not been associated with any pathology, the SNPs are, as part of a MPV-GPS implementation algorithm, leveraged to customize the optimal zone for the user. The relationship between genetic variants and hematological parameters is described, e.g., in Soranzo, Nicole, et al. “A genome-wide meta-analysis identifies 22 loci associated with eight hematological parameters in the HaemGen consortium,” Nature genetics 41.11 (2009): 1182-1190, which is incorporated herein by reference in its entirety.

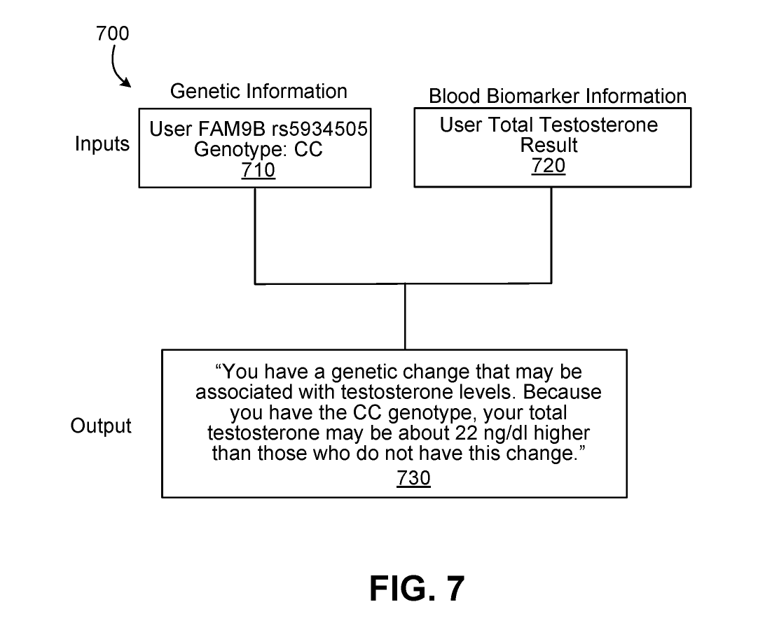

FIG. 7 is a block diagram showing an example of a process of generating an output by using genetic variants related to a CNV or an I/D rather than a simple SNP. For example, a male user with the rs5934505 SNP in the FAM9B CNV-insertion area and the “C” genotype for the SNP may have serum testosterone that is 22 ng/dL higher compared to “T” genotypes, and his optimal zone may thus be adjusted to reflect his genetic potential due to this variant.

Furthermore, rs4646994 can be used alone or as part of a genetic potential score algorithm in order to generate recommendations around a user's aerobic and/or anaerobic exercise capacity. rs4646994 is an insertion/deletion of an Alu repetitive element in an intron of the ACE gene. The relationship between genetic variants and exercise performance in atmospheric hypoxia is described, e.g., in Hennis, Philip J., et al. “Genetic factors associated with exercise performance in atmospheric hypoxia,” Sports Medicine 45.5 (2015): 745-761, which is incorporated herein by reference in its entirety. Other SNPs can be used to generate recommendations for a user's aerobic exercise capacity and/or endurance. Thus, in some implementations, at least 1, at least 2, at least 3, at least 4, at least 5, at least 6, at least 7, at least 8, at least 9, at least 10, at least 11, at least 12, at least 13, or all 14 SNPs selected from the group consisting of rs4646994, rs4343, rs1815739, rs1049305, rs1799722, rs12722, rs12594956, rs11549465, rs5219, rs4253778, rs2016520, rs7732671, rs660339, and rs2010963 can be used to generate the recommendation.

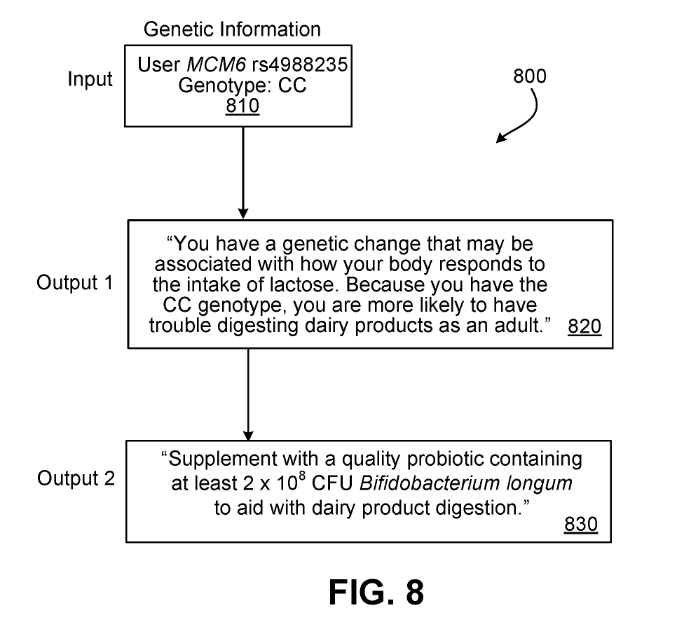

FIG. 8 is a block diagram showing an example of a process of generating, by the analysis engine 150, an output recommending probiotic supplementation for modulating the colonic microbiota in a subject. For example, a user with the “CC” genotype at rs4988235 is first informed that she/he is likely to be lactose intolerant in adult life 820. In such cases, a recommendation can be generated that individuals of this genotype are likely to tolerate increased consumption of dairy products with a Bifidobacterium-containing probiotic supplement 830. Such a recommendation can be based on a rule created using the relationship between lactose intolerance and genetic variants as described, e.g., in Enattah, Nabil Sabri, et al. “Identification of a variant associated with adult-type hypolactasia.” Nature genetics 30.2 (2002): 233-237, which is incorporated herein by reference in its entirety.

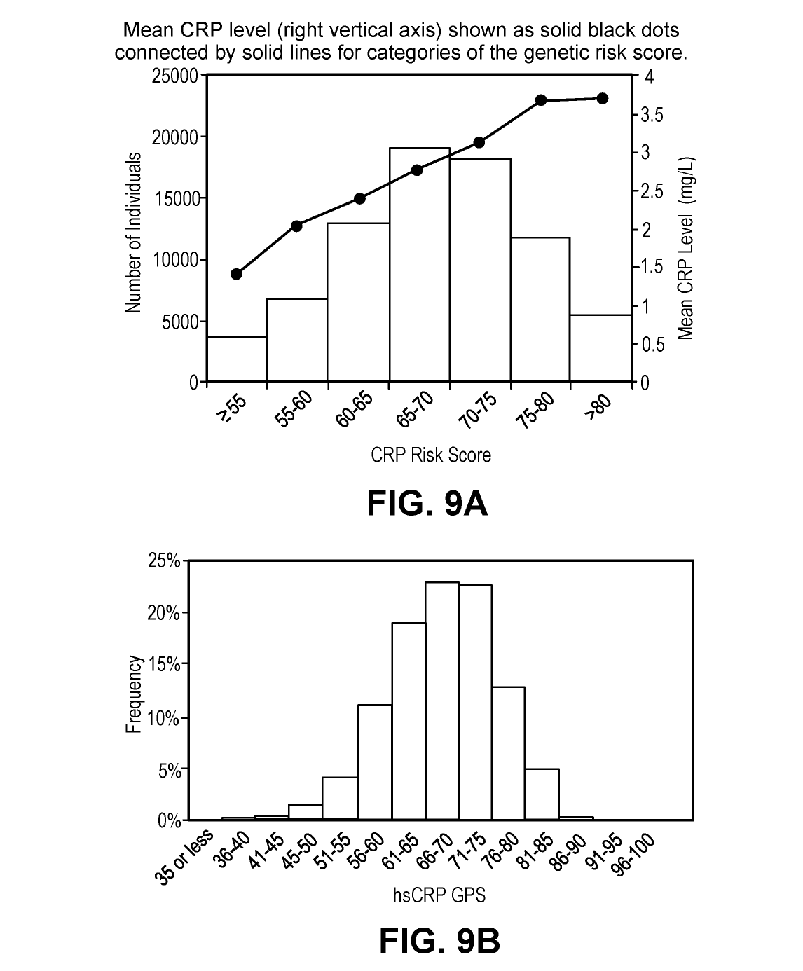

FIGS. 9A and 9B show the comparison result between the distribution of a weighted genetic score calculated from a theoretical population (FIG. 9B) and the distribution of CRP risk score derived from a real population dataset (FIG. 9A). The methods of calculating CRP risk score and the genetic variants are described, e.g., in Dehghan et al. “Meta-Analysis of Genome-Wide Association Studies in >80 000 Subjects Identifies Multiple Loci for C-Reactive Protein Levels, Clinical Perspective,” Circulation 123.7 (2011): 731-738, which is incorporated herein by reference in its entirety. 20 SNPs were selected from Dehghan et al. The theoretical population was generated by randomly assigning genotypes of each SNP to each subject in the theoretical population, so that the frequency for each genotype was consistent with the results of Hardy-Weinberg equilibrium. A weighted genetic score was calculated for each subject in the theoretical population. The distribution of the genetic score was then compared against the distribution of the CRP risk score. The results showed that the distribution of the weighted genetic score matches with the distribution of the CRP risk score, suggesting that the genetic scores calculated by the methods described herein correlate well with the actual CRP risk scores derived from a real population.

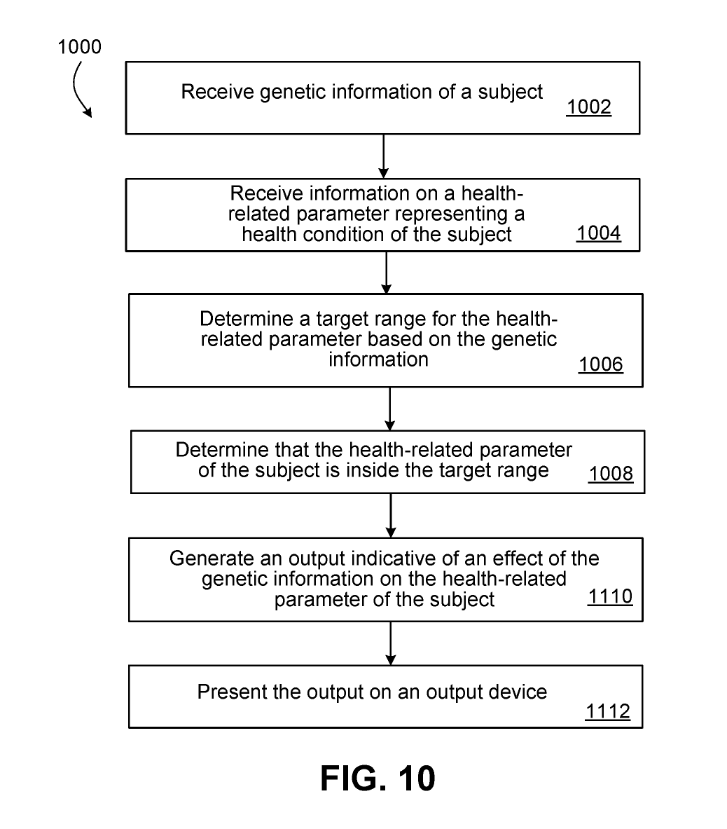

FIG. 10 describes an example process 1000 for generating and presenting one or more outputs based on genetic information about the user. In some implementation, at least a portion of the process 1000 may be executed by the analysis engine 150. Operations of the process 1000 includes receiving genetic information of a subject (1002), and receiving information on a health-related parameter representing a health condition of the subject (1004). The genetic information and the information on one or more health related parameters can be received in various ways, including for example, from a database storing such information and/or via user-input received using an appropriate user-interface. The genetic information can include, for example, genotypes of one or more SNPs, information of copy number variants, insertions/deletions, translocations, and/or inversions. The health-related parameters can include information on the levels of one or more biomarkers, one or more physiological markers, demographic information, lifestyle parameters such as smoking habits, exercise habits etc., or personal goals such as described above with reference to FIG. 1.

Operations of the process 1000 further includes determining a target range for the health-related parameter based on the genetic information (1006). The target range for a particular health-related parameter can be a range that is considered to be “optimal” for the particular subject based on the genetic profile of the subject. In some cases, the target range can be determined as a range that is indicative of general good health of the subject within the constraints of the genetic profile of the subject. The target range or optimal range can be determined individually for various health-related parameters (e.g., an optimal range of serum triglyceride level, an optimal range of fasting glucose, an optimal range of exercise level, etc.), and can be different from a reference range for the corresponding health-related parameter for the general population (e.g., average value of serum triglyceride level in the general population). In some implementations, the target range for a health-related parameter for the subject can be determined using one or more rules that associate the health-related parameter with the genetic information of a subject. For example, the target range can be determined based on a genetic score calculated using the genetic information. In one example, such a genetic score can be calculated by combining genetic information in weighted and unweighted combinations using one or more of the equations described above.

Operations of the process 1000 also includes determining that the health-related parameter of the subject is inside the target range (1008). The operations can also include generating, in response to determining that the health-related parameter of the subject is inside the target range, an output indicative of an effect of the genetic information on the health-related parameter of the subject (1010). The output can be presented on an output device (1012), using, for example one or more of the user interfaces described above with reference to FIGS. 2-4. In some implementations, the fact that the health-related parameter of the subject is inside the target range indicates that the health-related parameter is not a concern for the subject, and thus, in some cases, the subject is recommended to keep his/her current lifestyle, exercise routines, or diet. In some implementations, the operations of the process can also include determining that the health-related parameter of the subject is outside the target range, and generating, in response, a recommendation for affecting the health-related parameter of the subject. In some implementations, the fact that the health-related parameter of the subject is outside the target range may indicate that the health-related parameter may be improved, and accordingly, recommendations for modifying current lifestyle, exercise routines, and/or diet to improve the health-related parameter may be provided. For example, if serum triglyceride level of a subject is outside the target range (e.g., above the higher limit of the target range), a recommendation for more exercise and/or consuming less fat may be generated. In another example, if the serum 25(OH)D level for a subject is outside the target range (e.g., below the lower limit of the target range), a recommendation for taking vitamin D supplements, and/or more outdoor activities may be recommended.

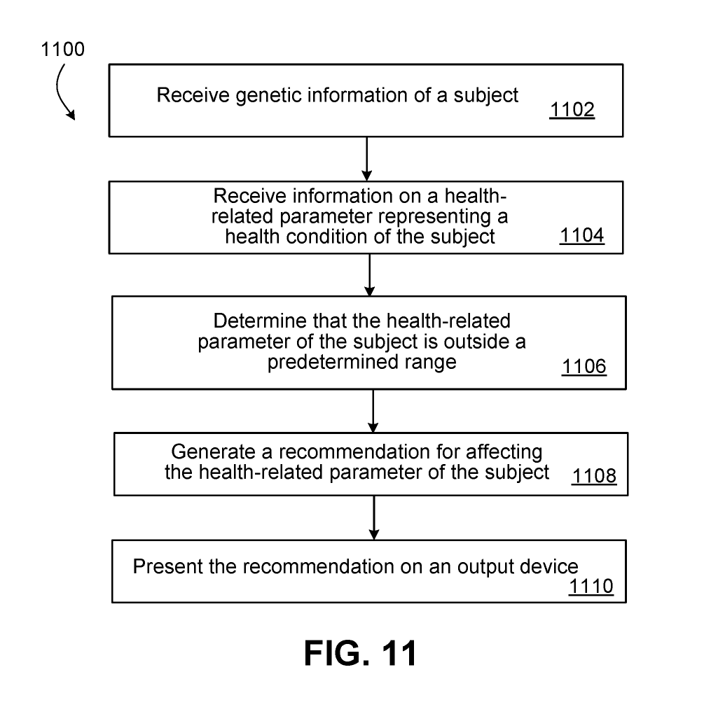

FIG. 11 shows a flowchart of an example process 1100 for generating and presenting recommendations based on genetic information and one or more health-related parameters of a subject. The process 1100 can be executed, at least in part, by the analysis engine 150 described above. Operations of the process 1100 includes receiving genetic information of a subject (1102), and receiving information on a health-related parameter representing a health condition of the subject (1104). The genetic information and the information on the health-related parameter can be received, for example, as described above with reference to FIG. 10.

Operations of the process 1100 also includes determining that the health-related parameter of the subject is outside a predetermined range (1106). In some implementations, the predetermined range can be substantially same as the target range or optimal range described above with reference to FIG. 10. In some implementations, the predetermined range can be a reference range in the general population (e.g., average value of serum triglyceride level in the general population). Operations of the process 1100 can also include generating, responsive to determining that the health-related parameter of the subject is outside the predetermined range, a recommendation for affecting the health-related parameter of the subject (1108). This can include, for example, generating an initial recommendation responsive to determining that the health-related parameter of the subject is outside the predetermined range, and modifying the initial recommendation based on a genetic score calculated using the genetic information. The genetic score can be calculated, for example, by combining the genetic information in weighted or unweighted combinations using one or more of the equations described above. Operations of the process 1100 further includes presenting the recommendation on an output device (1110), for example, using one or more user-interfaces.

The recommendation for affecting the health-related parameter of the subject based on his genetic profile may be generated in various ways. For example, if a subject has the GG genotype for the SNP rs2282679, the subject may be determined to be less responsive (e.g., 14% less responsive) to vitamin D supplements as compared to another representative population. Therefore, if the subject has a 25(OH)D level that is below the lower limit of the corresponding predetermined range, taking more vitamin D supplements (e.g., as compared to what is typical for the representative population), and choosing alternative ways to increase serum 25(OH)D level, e.g., increasing exposure to sunlight, may be recommended. Such recommendations can be generated based on one or more genetic scores calculated for the subject.

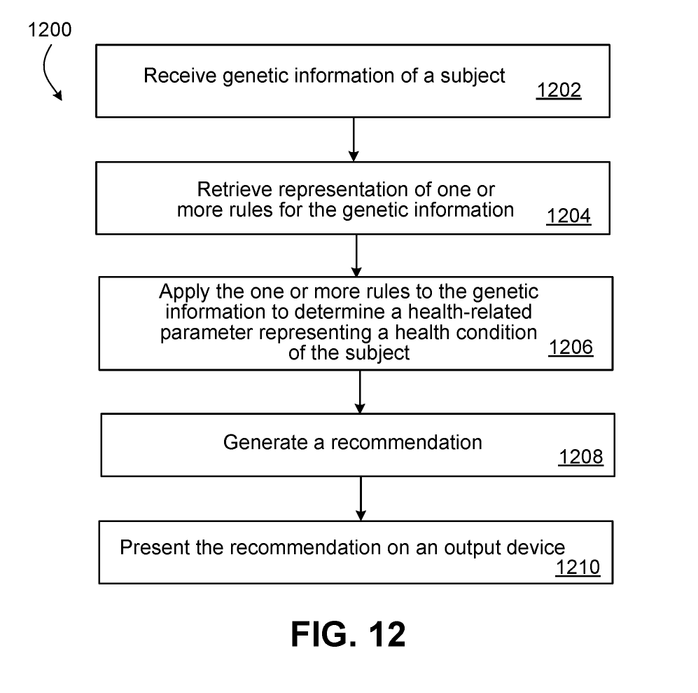

FIG. 12 shows a flowchart of an example process 1200 for generating one or more recommendations based on a subject's genetic profile. The process 1200 can be executed, at least in part, by the analysis engine 150 described above. Operations of the process 1200 includes receiving genetic information of a subject (1202), and retrieving representations of one or more rules for the genetic information (1204). The representations of the one or more rules can include computer-readable data that may be retrieved from a computer-readable storage device storing a database such as the rules database 160 described above with reference to FIG. 1. Operations of the process 1200 includes applying the one or more rules to the genetic information to determine a health-related parameter representing a health condition of the subject (1206), and generating, in response to the determined health-related parameter, a recommendation related to the health-related parameter (1208). In some implementations, the health-related parameter can include qualitative characteristics including, for example, predisposition to caffeine consumption, aerobic exercise capacity, sleep habits, etc.

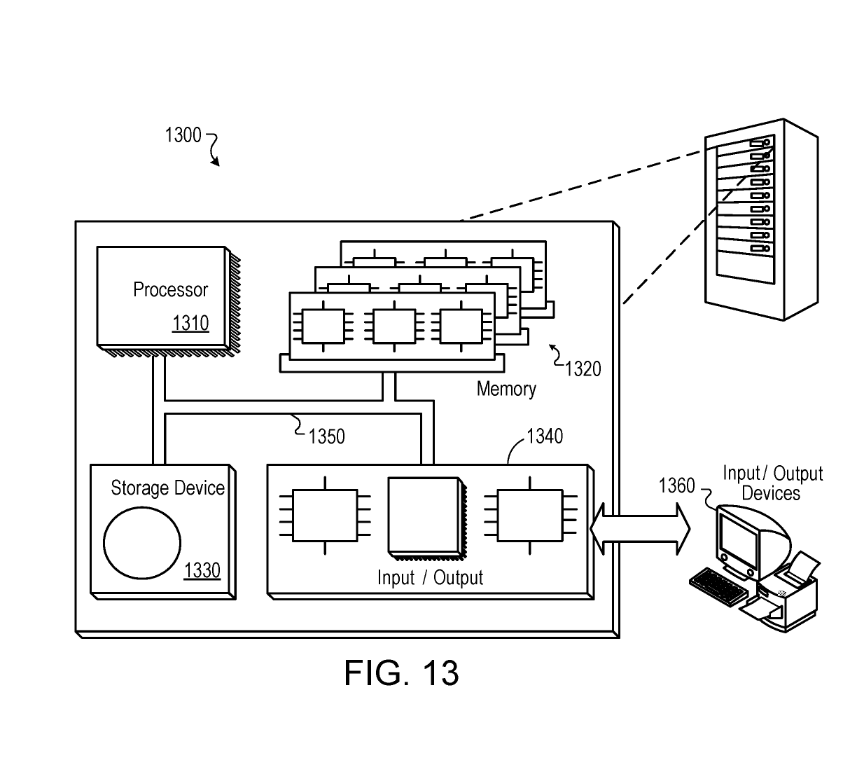

FIG. 13 is block diagram of an example computer system 1300 that may be used in performing the processes described herein. For example, the analysis engine 150 described above with reference to FIG. 1, can include at least portions of the computing device 1300 described below. Computing device 1300 is intended to represent various forms of digital computers, such as laptops, desktops, workstations, servers, blade servers, mainframes, and other appropriate computers. Computing device 1300 is further intended to represent various typically non-mobile devices, such as televisions or other electronic devices with one or more processers embedded therein or attached thereto. Computing device 1300 also represents mobile devices, such as personal digital assistants, touchscreen tablet devices, e-readers, cellular telephones, smartphones, smartwatches, and fitness tracking devices.

The system 1300 includes a processor 1310, a memory 1320, a storage device 1330, and an input/output module 1340. Each of the components 1310, 1320, 1330, and 1340 can be interconnected, for example, using a system bus 1350. The processor 1310 is capable of processing instructions for execution within the system 1300. In one implementation, the processor 1310 is a single-threaded processor. In another implementation, the processor 1310 is a multi-threaded processor. The processor 1310 is capable of processing instructions stored in the memory 1320 or on the storage device 1330.

The memory 1320 stores information within the system 1300. In one implementation, the memory 1320 is a computer-readable medium. In one implementation, the memory 1320 is a volatile memory unit. In another implementation, the memory 1320 is a non-volatile memory unit.

The storage device 1330 is capable of providing mass storage for the system 1300. In one implementation, the storage device 1330 is a computer-readable medium. In various different implementations, the storage device 1330 can include, for example, a hard disk device, an optical disk device, or some other large capacity storage device.

The input/output module 1340 provides input/output operations for the system 1300. In one implementation, the input/output module 1340 can include one or more of network interface devices, e.g., an Ethernet card, a serial communication device, e.g., an RS-232 port, and/or a wireless interface device, e.g., and 802.11 card. In another implementation, the input/output device can include driver devices configured to receive input data and send output data to other input/output devices, e.g., keyboard, printer and display devices 1360.

The web server, advertisement server, and impression allocation module can be realized by instructions that upon execution cause one or more processing devices to carry out the processes and functions described above. Such instructions can comprise, for example, interpreted instructions, such as script instructions, e.g., JavaScript or ECMAScript instructions, or executable code, or other instructions stored in a computer readable medium. The web server and advertisement server can be distributively implemented over a network, such as a server farm, or can be implemented in a single computer device.

Example computer system 1300 can include a server. Various servers, which may act in concert to perform the processes described herein, may be at different geographic locations, as shown in the figure. The processes described herein may be implemented on such a server or on multiple such servers. As shown, the servers may be provided at a single location or located at various places throughout the globe. The servers may coordinate their operation in order to provide the capabilities to implement the processes.

Although an example processing system has been described in FIG. 13, implementations of the subject matter and the functional operations described in this specification can be implemented in other types of digital electronic circuitry, or in computer software, firmware, or hardware, including the structures disclosed in this specification and their structural equivalents, or in combinations of one or more of them. Implementations of the subject matter described in this specification can be implemented as one or more computer program products, e.g., one or more modules of computer program instructions encoded on a tangible program carrier, for example a non-transitory computer-readable medium, for execution by, or to control the operation of, a processing system. The non-transitory computer readable medium can be a machine readable storage device, a machine readable storage substrate, a memory device, or a combination of one or more of them.

In this regard, various implementations of the systems and techniques described herein can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” “computer-readable medium” refers to a computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be a form of sensory feedback (e.g., visual feedback, auditory feedback, or tactile feedback); and

Attorney Docket No. 38891-0007001 input from the user can be received in a form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or a combination of such back end, middleware, or front end components. The components of the system can be interconnected by a form or medium of digital data communication (e.g., a communication network). Examples of communication networks include a local area network (“LAN”), a wide area network (“WAN”), and the Internet.

The computing system can include clients and servers. A client and server are generally remote from each other and typically interact through a communication network. The relationship of client and server arises by virtue of computer programs running on the respective computers and having a client-server relationship to each other.

Content, such as ads and GUIs, generated according to the processes described herein may be displayed on a computer peripheral (e.g., a monitor) associated with a computer. The display physically transforms the computer peripheral. For example, if the computer peripheral is an LCD display, the orientations of liquid crystals are changed by the application of biasing voltages in a physical transformation that is visually apparent to the user. As another example, if the computer peripheral is a cathode ray tube (CRT), the state of a fluorescent screen is changed by the impact of electrons in a physical transformation that is also visually apparent. Moreover, the display of content on a computer peripheral is tied to a particular machine, namely, the computer peripheral.

For situations in which the systems and methods discussed here collect personal information about users, or may make use of personal information, the users may be provided with an opportunity to control whether programs or features that may collect personal information (e.g., information about a user's calendar, social network, social actions or activities, a user's preferences, or a user's current location), or to control whether and/or how to receive content that may be more relevant to (or likely to be clicked on by) the user. In addition, certain data may be anonymized in one or more ways before it is stored or used, so that personally identifiable information is removed when generating monetizable parameters (e.g., monetizable demographic parameters). For example, a user's identity may be anonymized so that no personally identifiable information can be determined for the user, or a user's geographic location may be generalized where location information is obtained (such as to a city, ZIP code, or state level), so that a particular location of a user cannot be determined. Thus, the user may have control over how information is collected (and/or used) about him or her.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

A number of implementations have been described. Nevertheless, various modifications may be made without departing from the spirit and scope of the disclosure. For example, various forms of the flows shown above may be used, with steps re-ordered, added, or removed. Accordingly, other implementations may fall within the scope of the following claims.

1. A computer-implemented method comprising:
receiving genetic information of a subject;
receiving information on a health-related parameter representing a health condition of the subj ect;
determining, by one or more processing devices, a target range for the health-related parameter based on the genetic information;
determining that the health-related parameter of the subject is inside the target range;
generating, by the one or more processing devices in response to determining that the health-related parameter of the subject is inside the target range, an output indicative of an effect of the genetic information on the health-related parameter of the subject; and
presenting the output on an output device.

2. The method of claim 1, further comprising:
determining that the health-related parameter of the subject is outside the target range; and
generating, in response to determining that the health-related parameter of the subject is outside the target range, a recommendation for affecting the health-related parameter of the subject; and
presenting the recommendation on the output device.

3. The method of claim 1, wherein the target range is determined based on a genetic score calculated using the genetic information.

4. The method of claim 3, wherein the genetic score is compared against a distribution of the genetic score derived from a theoretical population.

5. The method of claim 1, wherein the genetic information comprises genotypes of one or more SNPs.

6. The method of claim 1, wherein the genetic information comprises information of copy number variants, insertions/deletions, translocations, and/or inversions.

7. The method of claim 1, wherein the health-related parameter represents a level of a blood biomarker.

8. The method of claim 1, wherein the target range for the health-related parameter is also determined by demographic information of the subject.

9. The method of claim 1, wherein the target range for the health-related parameter is also determined by a lifestyle parameter of the subject.

10. The method of claim 1, wherein the target range for the health-related parameter is also determined by exercise habit of the subject.

11. The method of claim 1, wherein the target range for the health-related parameter is also determined by personal goal of the subject.

12. The method of claim 1, wherein the health-related parameter is serum 25-hydroxyvitamin D (25(OH)D) level, and the genetic information comprises genotypes of rs2282679.

13. The method of claim 1, wherein the health-related parameter is serum triglyceride level, and the genetic information comprises genotypes of rs662799.

14. The method of claim 1, wherein the health-related parameter is serum LDL cholesterol level, and the genetic information comprises genotypes of rs964184.

15. The method of claim 1, wherein the health-related parameter is serum B12 level, and the genetic information comprises genotypes of rs602662.

16. The method of claim 1, wherein the health-related parameter is serum triglyceride level, and the genetic information comprises genotypes of one or more SNPs selected from the group consisting of rs1167998, rs673548, rs780094, rs2240466, rs10096633, rs122272004, rs439401, rs17321515, and rs16996148.

17. The method of claim 1, wherein the health-related parameter is fasting blood glucose level, and the genetic information comprises genotypes of one or more SNPs selected from the group consisting of rs7708285, rs11715915, rs17762454, rs2657879, rs340874, rs10747083, rs7651090, rs2302593, rs9368222, rs6943153, rs10814916, rs6072275, rs3829109, rs3783347, rs576674, rs4869272, rs11603334, rs174576, rs11619319, rs11607883, rs7903146, rs4502156, rs11708067, rs11039182, rs10811661, rs1280, rs983309, rs780094, rs10885122, rs2191349, rs11558471, rs6113722, rs16913693, rs2908289, rs560887, and rs10830963.

18. The method of claim 1, wherein the health-related parameter is mean platelet volume, and the genetic information comprises genotypes of one or more SNPs selected from the group consisting of rs10914144, rs11071720, rs11602954, rs12485738, rs1668873, rs2138852, rs2393967, rs342293, rs6136489, rs647316, rs7961894, and rs893001.

19. The method of claim 1, wherein the subject is a male, the health-related parameter is serum testosterone level, and the genetic information comprises genotypes of rs5934505.

20.-50. (canceled)

51. A system comprising:
a memory device; and
an analysis engine comprising one or more processing devices, the analysis engine configured to:
receive genetic information of a subject;
receive information on a health-related parameter representing a health condition of the subject;
determine, by one or more processing devices, a target range for the health-related parameter based on the genetic information;
determine that the health-related parameter of the subject is inside the target range;
generate, by the one or more processing devices in response to determining that the health-related parameter of the subject is inside the target range, an output indicative of an effect of the genetic information on the health-related parameter of the subject; and
present the output on an output device.

52. The system of claim 51, wherein the analysis engine is further configured to:
determine that the health-related parameter of the subject is outside the target range;
generate, in response to determining that the health-related parameter of the subject is outside the target range, a recommendation for affecting the health-related parameter of the subject; and
present the recommendation on the output device.

53. The system of claim 51, wherein the target range is determined based on a genetic score calculated using the genetic information.

54. The system of claim 53, wherein the genetic score is compared against a distribution of the genetic score derived from a theoretical population.

55. The system of claim 51, wherein the genetic information comprises genotypes of one or more SNPs.

56. The system of claim 51, wherein the genetic information comprises information of copy number variants, insertions/deletions, translocations, and/or inversions.

57. The system of claim 51, wherein the health-related parameter represents a level of a blood biomarker.

58. The system of claim 51, wherein the target range for the health-related parameter is also determined by demographic information of the subject.

59. The system of claim 51, wherein the target range for the health-related parameter is also determined by a lifestyle parameter of the subject.

60. The system of claim 51, wherein the target range for the health-related parameter is also determined by exercise habit of the subject.

61. The system of claim 51, wherein the target range for the health-related parameter is also determined by personal goal of the subject.

62. The system of claim 51, wherein the health-related parameter is serum 25-hydroxyvitamin D (25(OH)D) level, and the genetic information comprises genotypes of rs2282679.

63. The system of claim 51, wherein the health-related parameter is serum triglyceride level, and the genetic information comprises genotypes of rs662799.

64. The system of claim 51, wherein the health-related parameter is serum LDL cholesterol level, and the genetic information comprises genotypes of rs964184.

65. The system of claim 51, wherein the health-related parameter is serum B12 level, and the genetic information comprises genotypes of rs602662.

66. The system of claim 51, wherein the health-related parameter is serum triglyceride level, and the genetic information comprises genotypes of one or more SNPs selected from the group consisting of rs1167998, rs673548, rs780094, rs2240466, rs10096633, rs122272004, rs439401, rs17321515, and rs16996148.

67. The system of claim 51, wherein the health-related parameter is fasting blood glucose level, and the genetic information comprises genotypes of one or more SNPs selected from the group consisting of rs7708285, rs11715915, rs17762454, rs2657879, rs340874, rs10747083, rs7651090, rs2302593, rs9368222, rs6943153, rs10814916, rs6072275, rs3829109, rs3783347, rs576674, rs4869272, rs11603334, rs174576, rs11619319, rs11607883, rs7903146, rs4502156, rs11708067, rs11039182, rs10811661, rs1280, rs983309, rs780094, rs10885122, rs2191349, rs11558471, rs6113722, rs16913693, rs2908289, rs560887, and rs10830963.

68. The system of claim 51, wherein the health-related parameter is mean platelet volume, and the genetic information comprises genotypes of one or more SNPs selected from the group consisting of rs10914144, rs11071720, rs11602954, rs12485738, rs1668873, rs2138852, rs2393967, rs342293, rs6136489, rs647316, rs7961894, and rs893001.

69. The system of claim 51, wherein the subject is a male, the health-related parameter is serum testosterone level, and the genetic information comprises genotypes of rs5934505.

70.-100. (canceled)

101. One or more machine-readable storage devices storing instructions that are executable by one or more processing devices to perform operations comprising:
receiving genetic information of a subject;
receiving information on a health-related parameter representing a health condition of the subj ect;
determining, by one or more processing devices, a target range for the health-related parameter based on the genetic information;
determining that the health-related parameter of the subject is inside the target range;
generating, by the one or more processing devices in response to determining that the health-related parameter of the subject is inside the target range, an output indicative of an effect of the genetic information on the health-related parameter of the subject; and
presenting the output on an output device.

102. The one or more machine-readable storage devices of claim 101, further comprising instructions for:
determining that the health-related parameter of the subject is outside the target range; and
generating, in response to determining that the health-related parameter of the subject is outside the target range, a recommendation for affecting the health-related parameter of the subject; and
presenting the recommendation on the output device.

103. The one or more machine-readable storage devices of claim 101, further comprising instructions for:
calculating a genetic score based on the genetic information.

104. The one or more machine-readable storage devices of claim 103, further comprising instructions for:
comparing the genetic score against a distribution of the genetic score derived from a theoretical population.

105.-110. (canceled)
